(19) United States
(12) Patent Application Publication
(10) Pub. No.: US 2006/0008787 A1
(43) Pub. Date: Jan. 12, 2006

(54) Voice alert in dentistry

(76) Inventors: Robert Hayman, Los Angeles, CA (US); Eric P. Rose, Tarzana, CA (US); Christopher N. Quan, Quincy, MA (US); Nancy N. Quan, North Hills, CA (US)

Correspondence Address: BERGMAN KUTA LLP, P. O. BOX 400167, CAMBRIDGE, MA, 02140, US

(21) Appl. No.: 11/173,709

(22) Filed: Jun. 30, 2005

Related U.S. Application Data
(60) Provisional application No. 60/585,224, filed on Jul. 2, 2004.
(60) Provisional application No. 60/604,577, filed on Aug. 25, 2004.
(60) Provisional application No. 60/631,267, filed on Nov. 26, 2004.
(60) Provisional application No. 60/641,461, filed on Jan. 4, 2005.
(60) Provisional application No. 60/641,462, filed on Jan. 4, 2005.
(60) Provisional application No. 60/641,468, filed on Jan. 4, 2005.
(60) Provisional application No. 60/641,469, filed on Jan. 4, 2005.
(60) Provisional application No. 60/647,580, filed on Jan. 26, 2005.
(60) Provisional application No. 60/647,593, filed on Jan. 26, 2005.
(60) Provisional application No. 60/647,612, filed on Jan. 26, 2005.
(60) Provisional application No. 60/647,723, filed on Jan. 26, 2005.
(60) Provisional application No. 60/647,725, filed on Jan. 26, 2005.
(60) Provisional application No. 60/658,517, filed on Mar. 3, 2005.
(60) Provisional application No. 60/664,696, filed on Mar. 22, 2005.
(60) Provisional application No. 60/594,297, filed on Mar. 25, 2005.
(60) Provisional application No. 60/594,327, filed on Mar. 30, 2005.

(51) Int. Cl. G09B 23/28; G08B 1/00; A61B 5/117

(52) U.S. Cl. 434/263; 340/309.16; 433/229; 340/692; 368/10; 368/109; 368/251

(57) ABSTRACT

Dentistry equipment includes a voice alert device adapted to annunciate a status of the dentistry equipment or a process performed by the dentistry equipment. The voice alert device in various embodiments employs synthesized and recorded human voices.

### FIELD OF THE INVENTION

This invention relates to controls for dentistry applications. Specifically, this invention relates to voice alert in dentistry applications.

### BACKGROUND OF THE INVENTION

In the field of dentistry, many different dental procedures are carried out in a dental office that timing or depth reading is important. In tooth restoration and repair, dental cavities are often filled and/or sealed with compounds that are photosensitive, to visible and/or ultraviolet light. The compounds used in restoration or filling, commonly known as light-curable compounds, are placed within dental cavity preparations or onto dental surfaces and are cured by exposure to electromagnetic energy, such as visible or ultraviolet light, from a light-curing dental device. It is commonplace for this curing to be done with a hand held lamp system applied in close proximity to the filling compound after the compound has been placed inside the patient's tooth where the cavity once resided. A prescribed amount and type of illumination is needed in order for the compound to cure into a hardened state. Allowing a dentist to keep track of the amount of time that has transpired during this curing process helps to ensure that the curing process is complete and the composite resin is fully hardened.

In the field of dental whitening process, a patient's teeth are exposed to whitening agents and/or light employing the use of a chair side lamp system, especially when a light activatable whitening composition is used. The whitening composition is applied to the patient's teeth and a prescribed amount of time and/or type of illumination from a chair side lamp system is used. The timing is important in making sure that a correct amount of time and/or light energy is used.

In the field of endodontics, a root canal procedure involves the filing and reamering of the canal to remove as much dead pulp and other diseased material and debris from the canal as possible without actually reaching the apex. To keep track of the relative position of the apex in relationship to the cleaning and filing action is important to the efficacy and efficiency of the procedure.

In restorative dentistry, the curing lamps employed today primarily employ a system of audible beep tones to alert a dentist of the time that has transpired since illumination was started. Most often these lamps use predefined intervals between beep tones. Typically this may be five seconds between beep tones. In some systems, the dentist often has to count the beep tones to know how long the illumination light has been left on. Other systems may have a more complex beep tone system such as one beep for five seconds, two beep tones in rapid succession for ten seconds, three beep tones in rapid succession for fifteen seconds, etc.

Chair side teeth whitening also employs conventional beep tones or even stop watches. Again, the deciphering of the meaning of the beep, or which function or procedure is controlled by the stop watch may be confusing to a busy dental professional. Beep tones are used to indicate that a whitening lamp has completed its warm up cycle, nearly completed at this whitening cycle, and fully complete with a whitening cycle.

In root canal procedures, except for the use of a rubber stopper, there is no automatic indication of the process with relationship to the apex.

Other systems may include a display device that acts as a count down or count up timer. This type of system in dentistry requires a busy dental professional to view the display device.

All of the above mentioned methods for managing time or depth during dental procedures involve an extensive amount of manual tracking by a dentist or dental assistant. If the dental professional loses track of the amount of time a lamp has been applied to a patient, the dental procedure may have to be repeated to insure that either the curing or whitening process is complete. This cycling leads to unproductive lengthening of the procedure for the patient. Sometimes, dental professionals may even err on the side of applying too much light energy, leading to increased cost. In other processes, such as endodontics, cycling may not be possible.

### SUMMARY OF THE INVENTION

In view of the deficiencies identified above, it is desirable to have an easier and more efficient manner of alerting a dental professional of the cycle time or the relative depth. The present invention addresses these and other limitations of the prior art.

The present invention relates to a control system having a built-in voice alert system for alerting a dental professional of the time, depth or stage in a dental procedure. The dental procedures may include dental restoration, dental whitening and root canal procedures. The control system may also include a headphone or other private listening device, for example, so that only the dental professional will receive the voice alert. In one aspect, the private listening device may be a wireless listening device such as a wireless radio channeling device or an infrared channeling device.

In one embodiment of the invention, a dental light system includes a built-in electronic voice alerting system to alert the dental professional of the completion of a dental procedure. In one aspect, the light includes system a dental curing light. In another aspect, the light system includes a dental whitening light system.

In another embodiment of the invention, an apex locator includes a built-in electronic voice alerting system to alert the dental professional of the relative position of a root canal apex in relationship to the depth of reach during a root canal procedure.

In yet another embodiment of the invention, a lip retracting device includes a built-in electronic voice alerting system to alert the dental professional of the completion of an in office dental whitening procedure that may or may not utilize a light source.

In one aspect, the electronic voice alerting system utilizes an electronic voice generating circuit technology, similar to the technology used in electronic devices such as toys, cell phones, automobiles and other consumer electronics, but with novel message content that is directed to dental applications.

In still another embodiment of the invention, a dental lamp system includes an audible electronic voice alert system having a novel approach to tracking time during the above mentioned dental procedures and other similar dental procedures. This audible electronic voice alert system uses an electronic device with prerecorded time interval statements stored in the device. According to one embodiment, the alert system, in addition to giving indiscriminate beep tones at given intervals, or having a display screen displaying the stages of completion of a dental procedure, is also adapted to play a recorded voice that is generated when an electronic timer circuit is programmed to play the appropriate electronic voice count alert through an audio speaker in the device, or a recorded voice is generated to alert the dental professional of the depth of reach in a root canal procedure. In one aspect, the message played may include time intervals or depth of reach, and may be programmed and in some embodiments, re-programmed.

In a further embodiment of the invention, a dental lamp system having an electronic timer device is controlled by a microprocessor with an internal clock. The microprocessor receives a signal so as to know when a lamp is first turned on. At predefined intervals of, for example five seconds, the electronic voice chip sends a recorded audio signal to a speaker to announce elapsed and/or remaining time to the user. In one embodiment of the invention, the speaker is disposed within the light source. This process can be programmed to continue and announce. the time at ten second intervals when the voice chip releases a different recorded audio signal of “ten seconds”. Various time increments and corresponding audio signals can be programmed or selected according to the requirements of a particular dental procedure.

In yet a further embodiment of the invention, a dental lamp system includes a prerecorded audio stream that can be configured to play a unique alert message at the end of a procedure. The pre-recorded audio signal can include a message such as “procedure complete”, “end of a first cycle” when used in chairside whitening procedures, or similar phrase. Additionally, the system can be configured to give an instruction to the dental professional at certain times during the procedure. Exemplary messages may include prerecorded audio streams announcing, “the procedure is almost complete”, “please plan for the next step in the whitening process”, “whitening lamp warm up cycle complete,” And “the apex is approaching.” Numerous and various such voice alerts are possible and are intended to be within the scope of this invention.

In a yet still further embodiment, the invention, includes a dental instrument having a voice alert system in any of the above embodiments coupled to an electrical control device. The electrical control device may include a microprocessor and a switch such as an electromechanical switch or a solid state switch. In various embodiments, the electrical control device is adapted to both alert the dental professional that the end of the procedure or the apex is approaching, and also turn off the light output, or the power to the file or reamer to end the procedure when the predetermined time period has expired or where the prescribed distance to the apex is reached. This can further improve the efficiency and accuracy of a dental procedure and free the dental professional to take care of other matters within earshot of the voice alert system rather than having to hover around the patient or be close at hand to turn off the lamp, or to prevent any accidental reach of the apex in a root canal procedure.

### BRIEF DESCRIPTION OF THE DRAWINGS

FIG. 1 illustrates a curing light system of the present invention used in curing dental restorative materials.

FIG. 2 illustrates a lamp system of the present invention used in the dental whitening process.

FIG. 3 illustrates an apex locator system;

FIG. 4 illustrates a control module for an apex locator;

FIG. 5 illustrates, in block diagram form, an electronic voice alert system according to one embodiment of the present invention.

FIG. 5 illustrates another embodiment of the invention comprising an electronic voice chip with prerecorded time interval count alerts masked to the chip.

FIG. 6 illustrates an embodiment of the present invention comprising an electronic timer schematic with a microprocessor control (illustrates a voice alert system coupled to an on/off switch, yet another embodiment of the present invention).

FIG. 7 illustrates a prerecorded audio stream, another embodiment of the present invention.

FIG. 8 ilustrates a voice alert system including a personal audio transducer. (head phone, ear-bug)

FIG. 9 (FIG. 1a of D359:53360) simple one)

FIG. 10 (FIG. 1 in D359:41741, except there are no wings shown)

### DETAILED DESCRIPTION OF THE INVENTION

The detailed description set forth below is intended as a description of the presently exemplified embodiments provided in accordance with aspects of the present invention and is not intended to represent the only forms in which the present invention may be prepared or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to a control system having a built-in voice alert system for alerting a dental professional of the time, depth or stage in a dental procedure. The dental procedures can include dental restoration, dental whitening and root canal procedures.

Compact light sources, either hand held or mounted, can be used for curing composite materials used in tooth restoration or for tooth whitening processes.

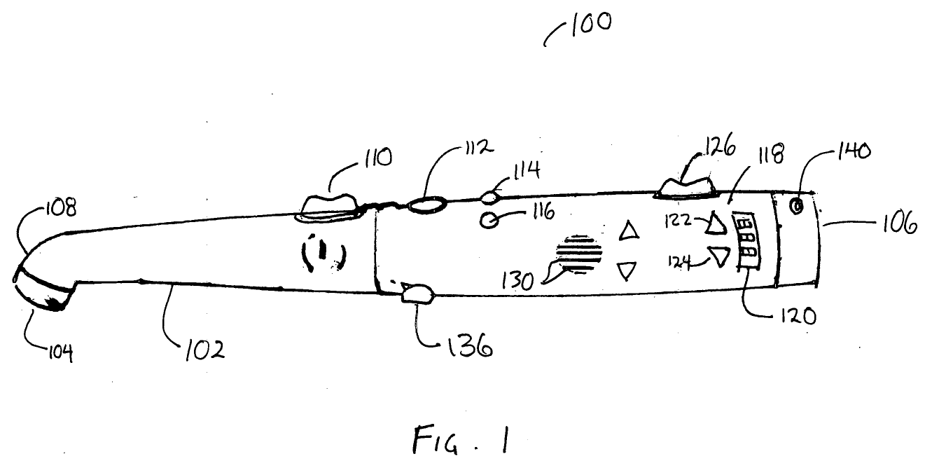

Light curable composite resin fillings have become the standard for filling dental cavities in dentistry today. A hand held lamp system 100, as illustrated in FIG. 1, is held in close proximity to a composite resin material located, such as within a cavity in a patient's tooth. The handheld lamp 100 subjects the composite material to a prescribed type and amount of light for a prescribed period of time in order for the composite resin to cure into a hardened state. Allowing a dentist to keep track of the amount of time that has transpired during this curing process is critical to ensure that the curing process is complete and the composite resin is fully hardened.

FIG. 1 shows a curing light 100 including voice alert according to one embodiment of the invention. The curing light 100 includes a housing 102. The housing 102 is adapted to contain and support a plurality of components including, for example a power module, such an electrical battery (not shown), a light source module 104, including, for example, a halogen arc light bulb, a light emitting diode, an incandescent light bulb, a fluorescent light bulb and a variety of control components such as, for example switches and indicators. In the illustrated embodiment, the housing 101 includes a proximal end 106 and a distal end 108. The curing light includes a main power switch 110, a light control switch 112 and battery status indicators (e.g., charging 114, battery charged 116).

According to one embodiment of the invention, the curing light 100 also includes a timer device 118 adapted to control a duration of illumination of the light source module 104. In the illustrated embodiment, the timer device an includes an elapsed-time and/or remaining time indicator 120, increment time 122 and decrement time 124 buttons a start and/or stop timer switch 126.

In a further aspect of the invention illustration as illustrated in FIG. 1, the curing light 100 includes an audio transducer. In the illustrated embodiment, the audio transducer disposed within the housing. The transducer is disposed adjacent to one or more apertures in an outer surface of the housing so that sound waves generated a by the audio transducer or received by the audio transducer can be readily coupled to an ambient medium, such as air. In one embodiment of the invention, the transducer is a miniature loud speaker.

According to various embodiments of the invention the miniature loudspeaker includes a moving coil speaker or a piezo-electric audio transducer device. In another embodiment of the invention, the audio transducer includes a microphone such as, for example, a moving coil microphone, a piezo-electric microphone, or a capacitive microphone such as, for example a condenser microphone, an electret microphone or a miniature integrated circuit microphone array. In still another embodiment of the invention, the transducer includes a single integrated device adapted to provide both loudspeaker and microphone functionality.

In the illustrated embodiment, the apertures 130 for the audio transducer device are shown disposed on a circumferential surface of the housing 102. In another embodiment of the invention, the apertures for the audio transducer device may be disposed at the proximal end 106 of the housing. In still another embodiment, the housing does not include apertures for sound transmission.

Instead the audio transducer is adapted to couple sound to the ambient air through a wall of the housing 101, so that no aperture is required. In still another embodiment, the wall of the housing forms an integral portion of the audio transducer so that, for example, the wall of the housing may include a specialized region formed of a material, such as a ceramic or a polymer having piezo-electric characteristics. Thus, the specialized region is adapted to receive a signal, such as an electrical signal, and responsively vibrate to produce an audio signal in the ambient air.

In the illustrated embodiment, the housing also supports a volume increment switch 132 and a volume decrement switch 134. According to this embodiment, the volume of audio signal generated by the audio transducer is controllable according to an operator input applied at the increment 132 and decrement 134 switches. In addition, according to one embodiment, a mute button 136 is supported by the housing 102. By application of a mechanical input to the mute button 136, a user can activate and deactivate an audio output and/or audio input of the system.

In still another aspect of the illustrated invention, the housing 102 supports an electrical coupling device such as, for example, an electrical plug receptacle 140. In one embodiment, the electrical receptacle is a standard subminiature electrical plug receptacle, as is known in the art.

The electrical plug receptacle is adapted to receive an electrical plug therewithin, or otherwise couple the curing light 100 to a remote audio transducer. In this way, an operator can receive audio signals from the curing light 100 and send audio signals to the curing light 100 by way of, for example, a headphone/microphone. Thus the audio signals produced by the curing light 100 can be received by the operator without the received signals being heard by, or disturbing a patient.

In operation, a curing light according to the invention is used to apply electromagnetic energy, such as, for example, ultraviolet radiation, to a dental composition. The dental composition may include, for example, one or more monomer materials and/or one or more polymer materials along with additional material such as fillers and reinforcing material. The dental composition is applied in an uncured state to a prepared dental surface such as, for example, a tooth or bone surface of a dental carie.

After application of the uncured material, the output electromagnetic energy the curing light is applied to the uncured composition. This electromagnetic energy allows or accelerates curing of the dental composition, so as to strengthen an adhesion between the dental composition and the dental surface as well as to harden the dental composition into a robust filling or adhesive.

The properties of the cured dental adhesive depend, in part, on the intensity and duration of the applied energy. Thus it is known to apply a curing light to a composition for curing in established time intervals. The duration of such time intervals may be measured using an external chronometer, such as, for example, a kitchen timer or wristwatch. The duration of the time intervals may also be measured and/or controlled by a timer device integrally formed as part of the curing light. The duration of time elapsed an/or a binary indication of process completeness may be provided by, for example indicator lights provided on the curing light.

According to the present invention, the curing light includes an audio system for providing audio output adapted to indicate a status of a curing process. For example, the audio system, in various embodiments, includes a timer and a sound generator. The sound generator receives control signals from the timer and responsively produces linguistic audio signals in a human-understandable language corresponding to an elapsed or remaining time in a particular curing cycle. For example, the audio signals indicate, according to one embodiment, how many minutes have elapsed since the light source 104 was illuminated.

In another embodiment, the audio signals indicate how many minutes remain before the illumination of the light source 104 is scheduled to be automatically or manually terminated. In yet another embodiment, the audio signals indicate elapsed or remaining time in terms of a fraction or percentage of a pre-programmed or adaptive processing cycle. Thus the sound generator generates a statement, according to one embodiment, that includes a phrase such as “processing is half done” or “curing is fifty percent complete.”

The language of the audio signals produced by the audio system may, in various embodiments, include any known human language, including combinations of languages and specialized or novel languages. In particular, the system may be adapted to respond in any known language such as English, Mandarin, French, Italian, and German, among others, or in concurrent or sequential combinations of languages.

In another embodiment, the information reported by the audio system includes measured or sensed information. Accordingly, in a particular embodiment of the invention, the curing light includes a sensor system adapted to detect a state of cure of a particular composition, such as by a variation in reflectivity with state of cure. Responsive to such a detected state of cure, the audio system makes an audio report such as “curing eighty percent complete.”

In another embodiment of the invention, the audio signals include information related to a status of the curing light system. Accordingly, in one embodiment, the audio system is adapted to provide messages indicating that the curing light is ready to operate such as “ready”, that the heat sink is approaching or has reached an operational temperature threshold; “heat storage capacity approaching—shut-down in two minutes” and similar messages.

In still another embodiment, a curing light according to the invention includes an audio system that can report on battery status; indicating, for example that the battery is 25 percent depleted, 50 percent depleted, 90 percent depleted, etc.

Additionally, in various embodiments of the invention, the audio system provides messages confirming the receipt of operator inputs. Thus when the operator presses the main power switch 110 to turn power on, the audio system responds with, for example, the message “power on” or “system now on.” If the operator presses the light control switch 112 with the light source module 104 in the un-illuminated state, the light source module 104 illuminates and the audio system reports “light on.” If the operator presses the light control switch 112 with the light source in the illuminated state, the light source module 104 turns off and the audio system reports “light off.”

In a further embodiment, the audio output signal is used to confirm an operator instruction or command, or to answer an operator question, as to for example system status, when the operator instruction or question is received by the system in audio format. In other words, in one embodiment, the system is adapted to receive voice input commands from an operator and to respond with informative responses in audio format.

In another embodiment of the invention, the audio system is adapted to report on a selection of a particular curing cycle or other programming choice. For example the audio system may identify a curing cycle, in a menu before selection, and/or after selection to confirm a selection that has been made. Thus a curing or other processing cycle may be identified according to its duration, according to a duty cycle, according to a particular generic or brand name of a composition to be processed, or according to any other parameter associated with operation of the curing light 100.

One of skill in the art will understand that the aforementioned reports and signals are not intended to represent a comprehensive list of messages. Rather, they are merely exemplary of the many states and signals of a dental curing light that are amenable to reporting by a linguistic audio system.

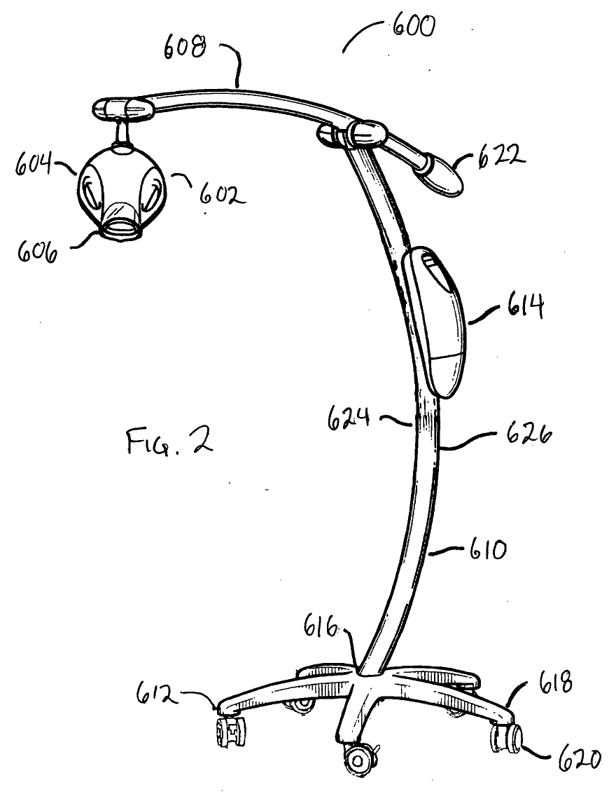

FIG. 2 shows, in perspective view, a lamp system according to the present invention. The lamp system may include a dental whitening, imaging or curing lamp system 600 according to one embodiment of the present invention. The lamp 600 includes a lamp head 602 having a lamp head housing 604 and a light guide 606. The lamp head 602 provides the light that, for example, activates a whitening substance or curing composite applied to a patient's teeth by directing the light through the light guide 606. This lamp system may be used in a dental office or a dental laboratory.

The lamp housing 604 and head 602 may be made of any polymeric material, for example, a polymer that can be molded or cast; or a metal or metallic alloy. Suitable polymers include polyethylene, polypropylene, polybutylene, polystyrene, polyester, acrylic polymers, polyvinylchloride, polyamide, or polyetherimide like ULTEM®; a polymeric alloy such as Xenoy® resin, which is a composite of polycarbonate and polybutyleneterephthalate or Lexan® plastic, which is a copolymer of polycarbonate and isophthalate terephthalate resorcinol resin (all available from GE Plastics), liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by reference)or combinations thereof.

In addition, any polymeric composite such as engineering prepregs or composites, which are polymers filled with pigments, carbon particles, silica, glass fibers, conductive particles such as metal particles or conductive polymers, or mixtures thereof may also be used. For example, a blend of polycarbonate and ABS (Acrylonitrile Butadiene Styrene) may be used for the lamp housing and head.

Generally, polymeric materials or composites having high temperature resistance are suitable.

Suitable metal or metallic alloys may include stainless steel; aluminum; an alloy such as Ni/Ti alloy; any amorphous metals including those available from Liquid Metal, Inc. or similar ones, such as those described in U.S. Pat. No. 6,682,611, and U.S. patent application Ser. No. 2004/0121283, the entire contents of which are incorporated herein by reference.

A liquid crystal polymer or a cholesteric liquid crystal polymer, one that can reflect rather than transmit light energy, may be used, either as a coating or as the main ingredient of the housing 604 and/or lamp head 602, to minimize escape of light energy, as described, for example, in U.S. Pat. Nos. 4,293,435, 5,332,522, 6,043,861, 6,046,791, 6,573,963, and 6,836,314, the contents of which are incorporated herein by reference.

The lamp head 602 is attached to a first end of a boom 608. The lamp head 602 is positionable with respect to the boom 608 and has a wide range of motion with respect to the end of the boom 608. The boom 608 is supported by a mast 610. In the illustrated embodiment, the boom 608 is pivotally mounted to the mast 610 at a point on the boom 608 closer to a second end of the boom 608 than the lamp head housing 604.

The boom 608 is adjustably positionable with respect to the mast 610. The boom 608 has both a rotational and a tilt range of motion with respect to the mast 610. A counterweight 622 on the second end of the boom 608 provides a counterbalance for the lamp head 602.

The mast 610 is attached to the base 612. In the illustrated embodiment, the mast 610 is fixed with respect to the base 612. In the embodiment shown, base 612 is a rolling base having a plurality of arms 618 extending radially from a center 616 of the base 612 where the mast 610 is attached.

The boom 608, mast 610 and base 612 may be fashioned out of any polymer or metal, such as those mentioned above for use in the lamp housing 604. Here, since the boom 608, mast 610 and base 612 are less likely to be subjected to any potentially high temperature environment, the suitable materials need not be of high temperature resistance. On the other hand, structural integrity is a more desirable feature.

In the illustrated embodiment, a caster wheel 620 is coupled to a respective distal end of each of the plurality of arms with respect to the center 616 of the base 612. The caster wheel 620 is adapted to contact, and thus to be supported by, a surface, for example, a supporting floor. In one embodiment of the invention, at least one of the caster wheels 620 includes a braking mechanism that prevents the caster wheel from rolling when the braking mechanism is in a locked position. In another embodiment of the invention, a plurality of caster wheels 620 includes the braking mechanism. In a further embodiment, a plurality of caster wheels 620 includes individual respective braking mechanisms.

The rolling base 612 enables the entire lamp system 600 to be positionable with regard to a patient in a dental chair. The rolling base 612 shown here is merely exemplary. Other types of rolling bases are contemplated within the scope of the invention. In addition, the mast 610 in other embodiments of the invention may be axially rotatable with respect to the base 612. According to one embodiment of the invention the mast 610 is curved and the curve accordingly defines a concave side 624 and a convex side 626 of the mast 610.

In the embodiment illustrated, a power pack 614 is attached to the mast 610 on the convex side 626. The power pack 614 includes controls for the lamp system 600.

The housing of the power pack 614, the rolling base 612 and rollers 620 may also be made out of any polymer or metal providing structural integrity, such as the materials mentioned above for use in the lamp housing 604. Here, since the rolling base 612 and rollers 620 are also not subject to. a potentially high temperature environment (unlike the power pack housing 614, the lamp housing 604 and the head 602), the suitable materials may not have the capability of high temperature resistance.

In operation, the lamp system 600 is positioned with respect to the patient in a dental chair (not shown). The location of the power pack 614 on the mast 610 enables the lamp system 600 to be operated whether the lamp system 600 is positioned to the right or to the left side of the patient. The curvature of the mast 610 enables the lamp system 600 to be positioned with respect to the patient such that the power pack 614 is located away from the patient making the lamp system 600 easier to operate.

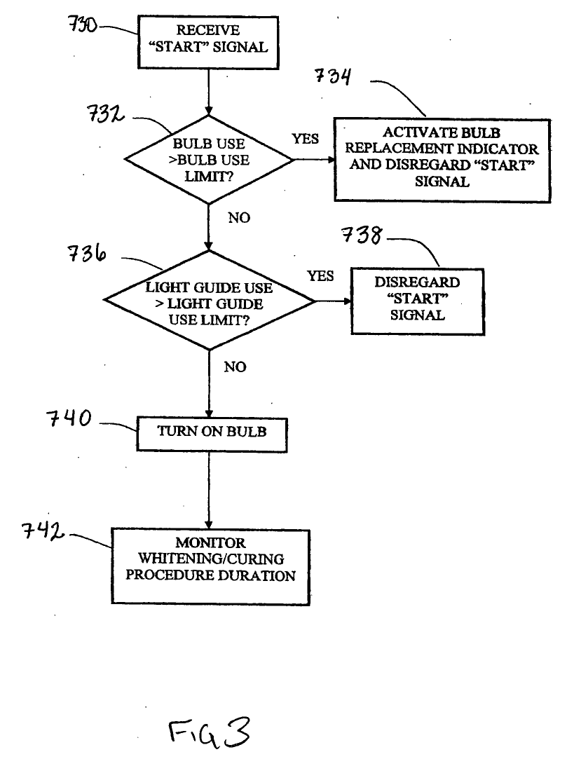

FIG. 3 is a flow chart showing one embodiment of the start process of the lamp system that is executed by the control system of lamp 600.

At step 730, the control system receives a “start” signal from the lamp controls 710. The “start” signal activates an initializing process that includes determining whether the light source 300 and light guide have reached their usage limits. The control system stores a light source usage limit, a light guide usage limit, and a whitening/curing procedure time limit that is divided into preselected time periods.

At step 732, the control system checks whether the light source 300 has been used longer than the light source usage limit stored in the control system. The light source usage limit is, for example, 100 hours. The control system monitors the time that the source 300 is on and adds this value to the amount of time accumulated from previous treatment procedures, if any. When the “start” signal is received from the lamp controls 710, the control system compares the accumulated light source on time with the light source usage limit. If the light source usage limit has been exceeded, the control system proceeds to step 734. If the light source usage limit has not been exceeded, the control system proceeds to step 736.

At step 734, the control system activates the light source replacement indicator 152 in the lamp head 102. In a first embodiment of the control system, the control system continues with the process of starting the lamp system 100. In this embodiment, the control system proceeds to step 736. In a second embodiment of the control system, the control system does not allow the lamp to be turned on. In this embodiment, the control system proceeds to step 738. In either embodiment, the control system is reset when the light source 300 is replaced.

At step 736, the control system determines whether the light guide usage has exceeded the light guide usage limit stored in the control system. The light guide usage limit is typically the amount of time of a single whitening or curing treatment. The light guide usage limit is, for example, sixty minutes. The control system, as mentioned above in step 732, monitors the time that the light source 300 is on. The control system writes the amount of time that the light source 300 has been on since the beginning of a treatment procedure to a recording device on the light guide 106. The recording device is, for example, a memory integrated circuit 246′. When the “start” signal is received from the lamp controls 710, the control system compares the light source “on” time stored on the recording device in the light guide 106 with the light guide use limit stored by the control system. If the light guide use limit has been exceeded, the control system proceeds to step 738. If the light guide use limit has not been exceeded, the control system proceeds to step 740.

At step 738, the control system disregards the “start” signal with regard to turning the light source 300 on. That is, the control system does not allow the lamp system 100 to operate if the light guide lifetime has expired. This portion of the control system acts to prevent the light guide from being reused. The light guide 106 is intended to be a single-use device to be discarded after each whitening or curing treatment.

At step 740, the control system starts the lamp (i.e. turns on the light source 300).

At step 742, the control system monitors the whitening or curing treatment procedure time. In this step, the control system monitors the time that the light source 300 is on. The monitoring procedure of the control system is described below with regard to FIG. 16.

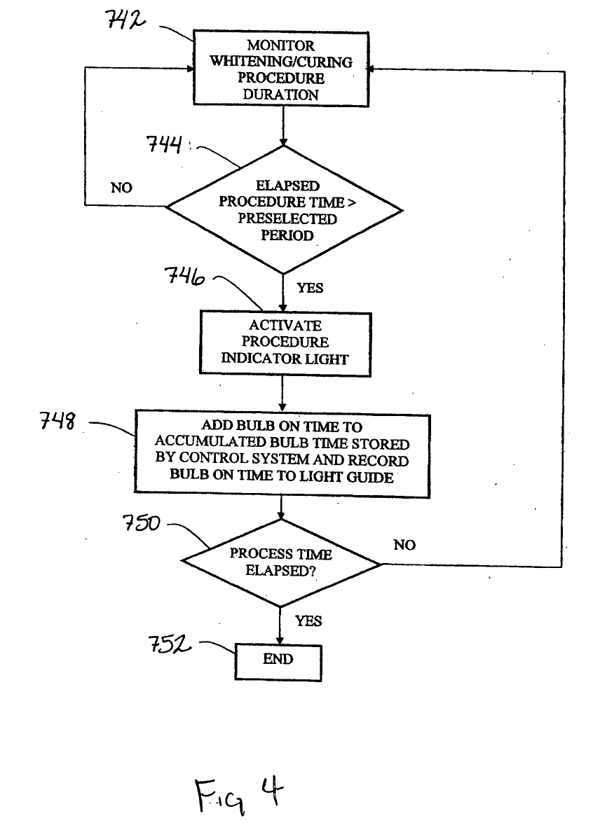

FIG. 4 is a flow chart showing one embodiment of the monitoring process of the lamp system that is executed by the control system illustrated in FIG. 14.

At step 742, the control system monitors the duration of the whitening or curing treatment, that is, the control system monitors the light source “on” time.

At step 744, the control system determines whether the elapsed procedure time has exceeded a preselected time period. Here, the preselected time period is some portion of the overall treatment time such as one quarter of the total treatment time. If the elapsed procedure time has not exceeded the preselected time period, the control system continues to monitor the treatment duration (step 742). If the elapsed procedure time does exceed the pre-selected time period, then the control system proceeds to step 746.

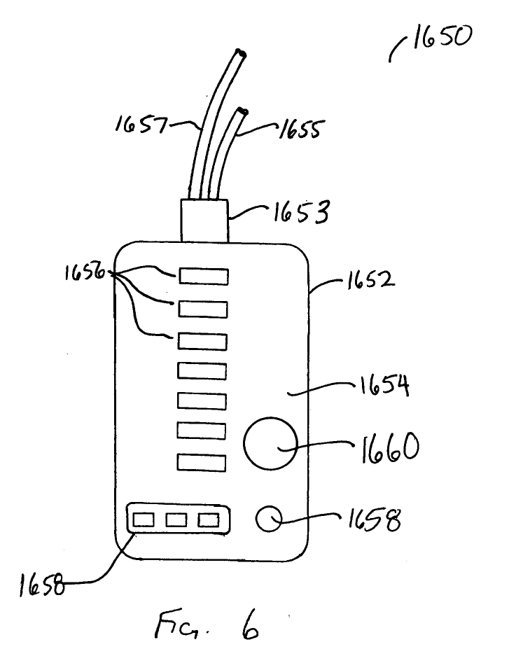

At step 746, the control system activates a procedure indicator light, for example one of the lighted indicators 150 described above with regard to FIG. 6. In one embodiment, the control system activates another lighted indicator 150 as each treatment portion time elapses so that if, for example, there are four lighted indicators, all four are lit at the end of the treatment procedure. In another embodiment, there is a single lighted indicator to indicate the time progression of the treatment. In this embodiment, the light indicator has varying flash rates to indicate the how much time has elapsed since the start of treatment. The control system then proceeds to step 748. In yet another embodiment, a voice alert system is used to alert the dental professional of the progress of the treatment, as described above.

At step 748, the control system adds the time that the light source has been on to the accumulated time that the control system has stored from previous treatment procedures, if any. The control system also writes the time that the light source has been on to the light guide recording device, such as the memory integrated circuit 246′. The control system then proceeds to step 750.

At step 750, the control system determines whether the overall process time has elapsed. The overall process time is the time duration of the whitening or curing treatment. If the overall process time has not elapsed, the control system returns to step 742, monitoring the whitening/curing duration. If the overall process time has elapsed, the control system proceeds to step 752.

At step 752, the duration of the whitening/curing treatment has elapsed and the control system turns off the light source 300.

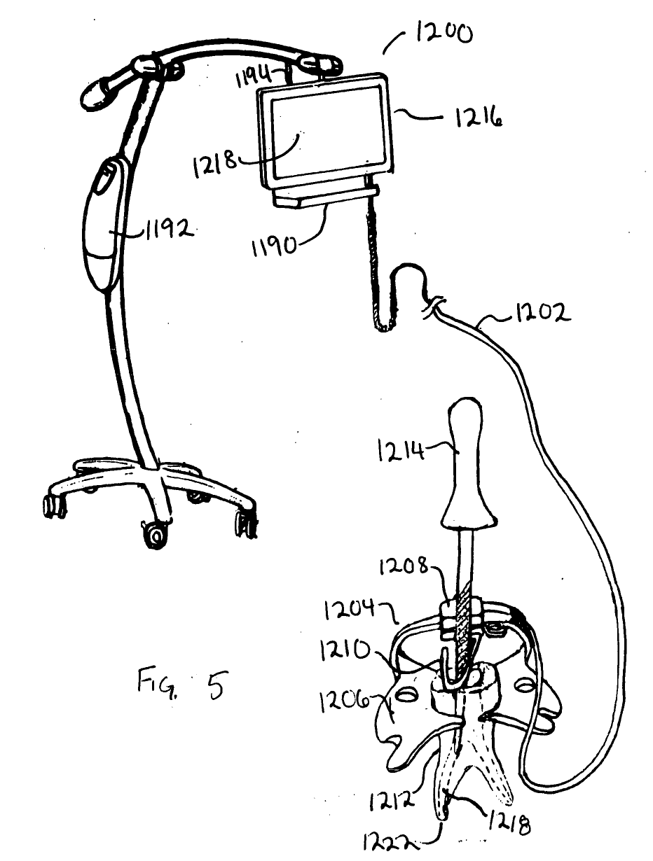

FIG. 5 shows another embodiment of the invention in which an apex locator system includes a linguistic audio system. The endodontic apex locator system 1200 includes a signal transmission medium such as a coaxial signal wire 1202. The signal wire 1202 is adapted to be coupled to an apex locator fixturing device 1204.

The apex locator fixturing device 1204 includes a support clamp portion 1206, an insulator portion 1208 and a contactor portion 1210. The support clamp portion 1206 is adapted to be removably but firmly coupled to a tooth 1212 of a dental patient. The support clamp portion 1206 is adapted to support the insulator portion 1208 which, in turn, is adapted to support the contactor portion 1210.

During the performance of a root canal procedure, an endodontic file, reamer, or other appropriate tool 1214 is used to excavate a pulp chamber and root of the tooth 1212. A surface of the tool 1214 comes into contact with the contactor 1210 and an electrical signal received from the signal wire 1202 is electrically coupled to the tool 1214.

The signal wire 1214 is coupled at a second end to a processing device 1216 that is adapted to generate the electrical signal and produce a measurement based on, for example, an impedance of the tool 1214 and tooth 1212 system.

In response to the measured impedance, or other signal, the processing system 1216 produces an image on a display screen 1218 of the system. In various embodiments, the image is textural and/or graphical, and represents a spatial location of the tool 1214 with respect to a root canal 1218 of the tooth 1212. Although the apex locator fixturing device 1204 is shown coupled to the balance of the apex locator system 1200 by signal wire 1202, a wireless apex locator fixturing system is also within the scope of the invention disclosed herewith.

In one aspect of the invention, the apex locator system of the invention is adapted to allow excavation and shaping of the root canal 1218 without perforation of the root wall or of the portion of the periodontal membrane located at the apex 1222 of the root.

In one aspect of the invention, the apex locator system includes a tray 1190 adapted to the convenient storage of various dental instruments and/or materials. According to one embodiment of the invention, the tray 1190 is readily removable to allow sterilization of the tray as in, for example, an autoclave.

In another aspect of the illustrated embodiment, the monitor screen 1218 of the system is pivotally and/or removably coupled to the boom 103 of the support structure at ball joint 902. In still another aspect of the illustrated embodiment, the apex locator system 1200 includes a power pack 1192.

According to one embodiment of the invention, the power pack supplies operative electrical power to the balance of the apex locator system by way of an electrical cable 1194. Still further embodiments of the invention include digital processing apparatus such as, for example, a microprocessor within the power pack 1192. The digital processing apparatus is adapted to control and process signals of the endodontic apex locator system.

In one embodiment the invention includes the combination of a support structure 100 with an endodontic apex locator system such as that disclosed in U.S. patent application Ser. No. 60/594,388 the disclosure of which is herewith incorprorated by reference in its entirety.

FIG. 6 shows a further embodiment of an apex locator system according to the invention including a hand-held control module. The illustrated embodiment 1650 includes a visual indicator device and a linguistic audio system according to the invention. The illustrated embodiment includes a housing 1652 having an upper surface 1654. The housing also includes, according to one embodiment, a coupling 1653. The coupling 1653, in the illustrated embodiment, supports a first signal wiring 1655 adapted to be coupled to the probe, and a second signal wire 1657 adapted to be coupled to a return contact according to the invention.

In one aspect of the illustrated embodiment, the upper surface 1654 includes a plurality of lights 1656 such as, for example, light emitting diodes. In other embodiments of the invention, the lights 1656 are gas discharge lamps, such as, for example neon lamps, incandescent lamps, fluorescent lamps, arc lamps, or combinations of the foregoing. In still another embodiment of the invention, the light 1656 is replaced by, or supplanted by, non-illuminated devices such as reflective liquid crystal indicator devices. Also shown is an audio transducer 1660. The audio transducer is adapted to couple audio voice signals to the ambient air as described above in relation to FIG. 1.

According to one embodiment of the invention, an illumination state of the lights 1656 corresponds in analog fashion to a measured proximity between the probe and an apex of a subject tooth. According to one embodiment of the invention the lights and 1656 are arranged in a linear array, and are adapted to be illuminated successively starting with a first light at one end of the array and proceeding to the last light at the other end of the array. Thus illumination of the first light indicates an extreme distance between the probe and the apex of the subject tooth, and illumination of the last light indicates an extreme proximity between the probe and the apex of the subject tooth.

According to one embodiment of the invention, the lights are adapted to remain illuminated once illuminated until a corresponding level of proximity has again been passed. Accordingly, the set of lights indicating a greater proximity than a current position of the probe with respect to the apex of the tooth forms a “bar” having a longitudinal dimension that varies with proximity to the apex of the tooth.

Another embodiment of the invention, one or more lights 1656 proximate to one end of the set of lights are adapted to emit a different wavelength of light than the light emitted by one or more other lights of the set of lights 1656.

Accordingly, for example, green lights may be illuminated when the probe is far from the apex of the tooth. Yellow lights may be illuminated when the probe is at an intermediate distance with respect to the apex of the tooth, and red lights may be illuminated when the probe is near to the apex of the tooth.

One of skill in the art will appreciate that other arrangements of lights, or other visual indicators, are found in various embodiments of the present invention. For example, the lights may be arranged in a nonlinear sequence, such as a curved arrangement, a circular arrangement, a spiral arrangement, or any other arrangement including combinations of the foregoing arrangements. Also, the lights may be arranged in a two-dimensional array, or even a three dimensional array, depending on the requirements of a particular application.

According to still another embodiment of invention, the apex locator system housing 1652 includes a multi-pixel indicator such as, for example, an LED display screen, a gas discharge display screen, or other display screen adapted to present status information in a visual representation. In various embodiments, the multi-pixel indicator is present alone, or in combination with other lights 1656 or visual indicators.

In still another embodiment of the invention, the apex locator does not include a visual indicator, but communicates with a user purely by way of audio signals, including linguistic audio signals.

In a further aspect of the illustrated embodiment, the upper surface 1654 includes an on/off switch 1658. The on/off switch is, in various embodiment, a pushbutton switch, a toggle switch, a sliding switch, a rotary switch, a key switch, or any other sensing device appropriate for on/off control of activation state of the apex locator system 1650.

According to another embodiment of the invention, the apex locator system 1650 includes an audible indicator providing information related to status of the system, including information related to proximity between the probe and an apex of a subject tooth. The audible indicator may provide information in the form of tone signals, buzzes, music, and synthesize or recorded voice signals.

In still another aspect of the illustrated embodiment, the upper surface 1654 of the apex locator system 1650 includes a battery status indicator 1658.

The instrument can also be fitted with electronic and digital capability along with a USB connector for connecting to a USB port of a computer equipped with or having capability for connection to medical practice management software, to enable the recording of the root canal procedure.

For example, according to one embodiment of the invention, a chronological record is made of a plurality of proximity values corresponding to sensed distances between the probe and an apex of a subject tooth. Thus, a plurality of distance readings may be made by the apex locator system 1650. According to one embodiment of the invention, this plurality of distance readings is digitized by an analog to digital converter to produce a corresponding plurality of digital values. The plurality of digital values is stored on a recording medium for future reference.

According to one embodiment of the invention, the recording medium includes a semiconductor memory device such as, for example, a flash memory device, a magnetoresistive memory device, a random access memory device such as a static random access memory device, an optical memory device, or any other appropriate memory storage medium as is known in the art.

According to another embodiment of the invention, the above-mentioned storage of proximity data onto a recording medium includes transmission of the from a processor within the housing 1652 into a storage device by way of, for example a Universal Serial Bus (USB) port.

In another embodiment of the invention, proximity data is transmitted, by way of storage on a storage medium, by optical communication channel, by electrical communication channel, or by wireless syndication channel, to a patient information system. According to one embodiment of the invention, the proximity information is stored in a record of the patient information system that corresponds to a particular patient. In addition, the record may include information correlating the proximity information to a dental practitioner, or to various other factors such as equipment.

According to one embodiment of the invention, the store proximity to his available for future reference for use in relation to, for example, future and procedures, or for demonstrating, for example, an absence of injury to the patient.

One of skill in the art will appreciate that conventional practice involves manual or electronic probing of a tooth to establish depth of penetration, followed by application of a rubber collar 101, as shown in FIG. 1, to a dental instrument to indicate a limit of further excavation. The above-described electronic system will facilitate and improve the efficiency of root canal procedures by reducing or eliminating time otherwise required for probing and application of rubber collars. Instead, feedback, such as audio (or other sensory e.g., visual, tactile) feedback is provided to a dental practitioner in real time. The feedback is provided in real time, and indicates to the dental practitioner the characteristics and consequences of an ongoing root canal excavation.

When a whitening composition is applied to a patient's teeth in a dental office without employing a light source, dental professional also tracks the amount of time the composition stays on the teeth. A voice alerting system may be incorporated in, for example, a lip retracting device used in the process.

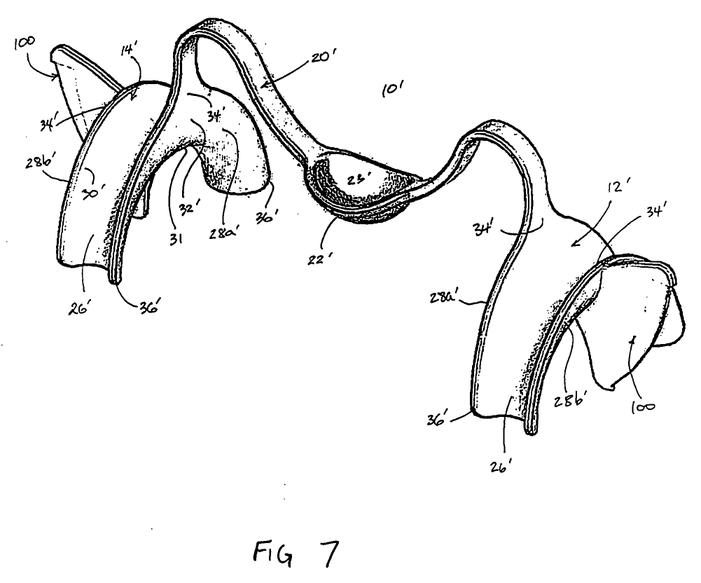

In FIG. 7, a lip retracting device 10 for retracting a user's upper and lower lips (herein “lips”) for facilitating examination and/or treatment of the mouth and/or teeth. When used, the retractor 10′ draws back the lips, which retracts the cheeks, to expose the mouth so that a health care professional can more easily see the teeth and work on the teeth and/or mouth.

The retractor 10′ includes two spaced apart channel retainers 12′ and 14′, also known as flanges, for retaining two corresponding portions of the lips for examination and/or treatment of the mouth or teeth. When used, the retractor 10′ draws back the lips, which retracts the cheeks, to expose the mouth so that a health care professional can more easily see the teeth and work on the teeth and/or mouth.

The two channel retainers 12′ and 14′ are for retaining the ends of the lips, approximately where the upper and the lower lips intersect. More particularly, the two channel retainers or flanges 12′ and 14′ are adapted to cup the lips and bias them open to expose the teeth for treatment and/or examination.

A resilient member 20′ is incorporated in the retractor 10′ to interconnect the two channel retainers 12′ and 14′ together and to function as biasing means. The resilient member 20′ has two arches, one on either side of the center portion 22′. The resilient member 20′ can be formed as a single piece integrally molded or attached to the inside side walls 28a′ of the channel retainers 12′ and 14′, or it can be formed in two halves separately connected to the mid-portion 22′, also integrally molded or attached to the inside side wall 28a′ of the channel retainers 12′ and 14′.

In the ready position (before insertion of the retractor into the mouth), the resilient members 20′ are arched outwardly with respect to the center portion of the retractor 10′. As further discussed below, when the retractor 10′ is inserted into the mouth and the two channel retainers 12′ and 14′ cup respective portions of the lips, the resilient members 20′ provide a retractive force to retract the lips radially outwardly for examination and/or treatment. This retractor is especially useful for the whitening process.

An optional tongue retainer 22′ can also be approximately centrally positioned relative to the two channel retainers 12′ and 14′. The tongue retainer 22′ of the present embodiment can also include a trough 23′. Further, it can be integrally formed on the mid-portion of the resilient member 20′ and thus be attached to the channel retainers 12′ and 14′ via resilient member 20′. When incorporated, the tongue retainer blocks the tongue and limit the tongue to the back vicinity of the mouth, thus enabling access to the lingual portion or back of the teeth for examination and/or treatment. In short, the tongue retainer is configured to minimize interference by the tongue during treatment and/or examination by a health care professional.

In this embodiment, the resilient member 20′ acts not only to connect the channel retainers and to bias them, but also to connect the tongue retainer to the channel retainers. If the tongue retainer 22′ is not incorporated, the resilient member 20′ would simply extend from one channel retainer 12′ to another channel retainer 14′ at a substantially uniform width.

The channel retainers 12′ and 14′ resemble a curvilinear c-channel in that they include an arcuate race 26′ and two channel side walls 28a′, 28b′. The channel side walls 28a′, 28b′ resemble a bell shape and include a maximum wall dimension at approximately the mid-point 34′ and two smaller tapered tips 36′ at the ends thereof. In one embodiment, the inside side wall 28a′, which is intraoral as further discussed below, is slightly larger relative to the outside side wall 28b′. However, the relative dimensions can be reversed or can be the same without deviating from the functionality of the lip retractor 10′.

The side channel retainers 12′ and 14′ further include an interior surface 30′ and an exterior surface 32′. The arcuate race 26′ comprises a radius of curvature 31′ adapted to mimic the curvature of the side of the lips when the lips are in the opened position. Because this curvature may vary depending on the size and age of the user or patient, the retractor 10′ may be implemented with varying radius of curvatures 31′ to fit the varied shape of the particular user/patient.

The arcuate race 26′ may also include an irregular curvature or two or more different radiuses of curvatures. For example, the lower region 38′ of the radius of curvature 31′ may have a larger radius than the upper region 40′ or vice versa. If implemented, the irregular curvature can vary the amount of retraction of the portion of the lip that is seated within the arcuate race to vary the amount of retraction between those portions of the lip.

The retractor 10′ may also be made by injection molding or casting a thermoplastic material such as polypropylene, polyethylene, polystyrene, polyester, polycarbonate or the like. More preferably, the retractor 10′ is made by injection molding pigmented polypropylene and is opaque white or colored having a smooth finish.

Additionally, FIG. 7 also shows two wing-like flanges 100 extending from the outside side wall 28b′ of the channel retainers 12′, 14′. The wing-like flanges 100 can be molded or cast integrally with the channel flanges or retainers 12′ and 14′. The wing-like flanges are designed for fitting the retractor 10′ to the slots formed on a cone section of an output port or light guide of a lamp source used in a teeth whitening process, or to the slots in any examining cone. The examining cone may include a voice alert system, such that when the retractor 10′ is fitted into the cone, a timing device is actuated. When the time period is concluded, the voice alert system alerts the dental professional of the progress or completion of the procedure.

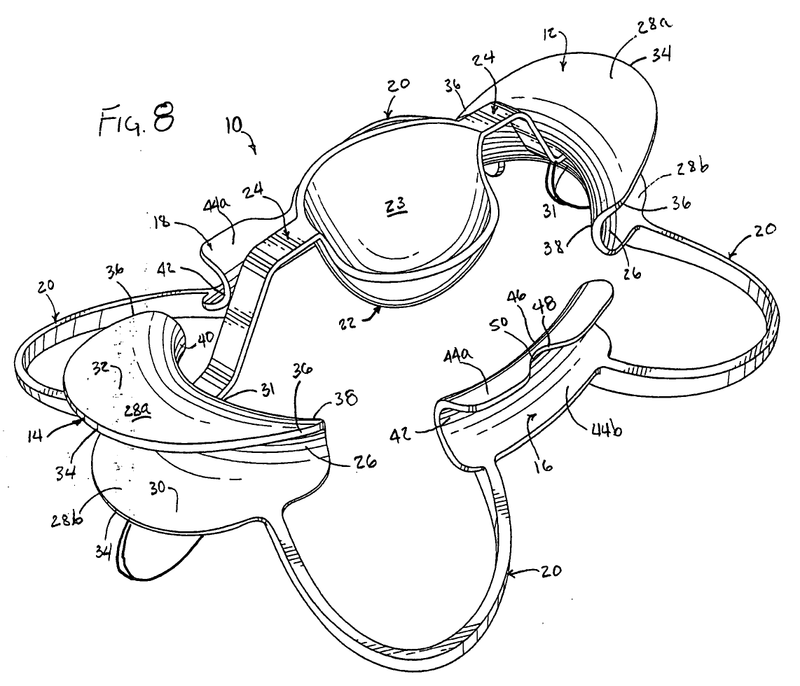

In another embodiment, as shown in FIG. 8, the lip retractor 10 may include four channel retainers including two side channel retainers 12, 14 for retaining the ends of the lips, approximately where the upper and the lower lips intersect, and two lip channel retainers 16, 18 for retaining the mid-section of the upper and lower lips. More particularly, the four channel retainers or flanges 12, 14, 16, 18 are adapted to cup the lips and bias them open to expose the teeth for treatment and/or examnination.

The retractor 10 also includes a plurality of resilient members 20, for example, four resilient members, which are incorporated in the retractor 10 to interconnect the four channel retainers 12, 14, 16, 18 together and to function as biasing means. Each channel retainer includes a race, an inside side wall, and an outside side wall, and wherein each resilient member is integrally molded to two outside side walls of two adjacent channel retainers and includes an arch.

An optional tongue retainer 22 is shown approximately centrally positioned relative to the four channel retainers 12, 14, 16, 18. The tongue retainer can also be positioned asymmetrically about the two channel retainers 16 and 18. The tongue retainer 22 comprises a trough 23 and is attached to two channel retainers 12, 14 by a pair of secondary resilient members 24.

When incorporated, the tongue retainer 22 and the secondary resilient members 24 cooperate to block the tongue and limit the tongue to the back vicinity of the mouth, thus enabling access to the lingual portion or back of the teeth for examination and/or treatment. In short, the tongue retainer is configured to minimize interference by the tongue during treatment and/or examination by a health care professional.

Two wing-like flanges 100 extending from the outside side wall 28b′ of the channel retainers 12′, 14′. The wing-like flanges 100 can be molded or cast integrally with the channel flanges or retainers 12′ and 14′. The wing-like flanges are designed for fitting the retractor 10′ to the slots formed on a cone section of an output port or light guide of a lamp source used in a teeth whitening process, or to the slots in any examining cone. The examining cone may include a voice alert system, such that when the retractor 10′ is fitted into the cone, a timing device is actuated. When the time period is concluded, the voice alert system alerts the dental professional of the progress or completion of the procedure.

The wing-like members or flanges can be made of the same material as the rest of the retractor or of a more sturdy polymeric material or composite. Additionally, it can also be opaque or colored even if the rest of the retractor maybe colorless or clear.

The voice alert system, whether used in curing composites, whitening teeth, or locating the apex of a root canal, may employ an electronic voice generating circuit technology.

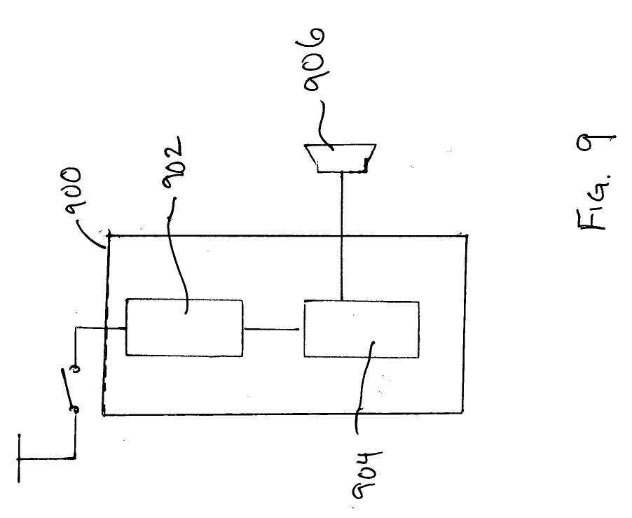

FIG. 9 shows a schematic for an audible electronic voice alert system. The alert system includes a novel approach to tracking time during, for example, either dental bleaching or tooth restoration procedures. In one embodiment, this audible electronic voice alert system uses an electronic voice chip 900 with prerecorded time interval count alerts masked to the chip. The voice chip 900 comprises an electronic timer circuit 902. A dedicated microcontroller 904 coupled to an audio transducer 906, such as a speaker, is adapted to produce synthesized voice announcements in response to output signals from the timer circuit 902. Instead of having indiscriminate beep tones at given intervals, as in prior art systems, a recorded voice is generated when an electronic timer circuit is programmed to play the appropriate electronic voice count alert through an audio speaker in the curing light or lamp system.

In another embodiment, the microprocessor comprises an internal clock. When the lamp is first turned on, the clock starts counting the time. At predefined intervals, for example five seconds, the electronic voice chip sends a recorded audio stream to a speaker in the lamp system, which would then announce, “five seconds.” This process could continue and at ten seconds, the voice chip would release a different recorded audio stream, “ten seconds.” This process could be repeated to announce the counter alert and play any number of prerecorded audio streams.

In one embodiment, the prerecorded audio stream could be configured to play a unique alert message at the end of a procedure such as, “procedure complete” or similar. Additionally, the system could even be configured to give instruction to the dental professional at certain points during the procedure. Examples may include prerecorded audio streams announcing, “the procedure is almost complete, please plan for the next step in the whitening process”, or “whitening lamp warming up cycle complete.” Numerous such voice alerts are possible and are within the scope of this invention.

In a further embodiment, the lamp system comprises an on/off circuit, coupled to the timer. At the end of a cycle, the voice chip could play the unique prerecorded “procedure complete” message, at which time the timer turns of the lamp system to end the procedure. This could add to the efficiency of the dental office.

While each of the embodiments described above includes a linguistic audio system, the linguistic audio systems are not necessarily of a single design or arrangement. For example in one embodiment, the linguistic audio system includes a memory device adapted to store pre-recorded message information, constituting recordings of an actual human voice, and a device adapted to convert the stored information to audio playbacks of the actual human voice.

In some embodiments, the message information includes recordings of complete messages. In other embodiments, the message information includes recordings of phrases, single words, or parts of words and phonemes. In such systems an audio playback device forms complete messages by combining selected ones of the phrases, single words, parts of words and phonemes.

In some embodiments of the invention, message information is stored in analog data format. In other embodiments of the invention, data is stored in digital data format. In still other embodiments of the invention, hybrid information is stored using digital and analog storage methodologies.

In still other embodiments of the invention, voice signals are synthesized directly from linguistic rules and data without the use of recorded voice information. In such systems, data representing anticipated message words is stored for retrieval and speech synthesis.

In still other embodiments of the invention, voice signals are created using both stored and synthesized speech components, where the stored speech components may include analog or digital stored speech components.

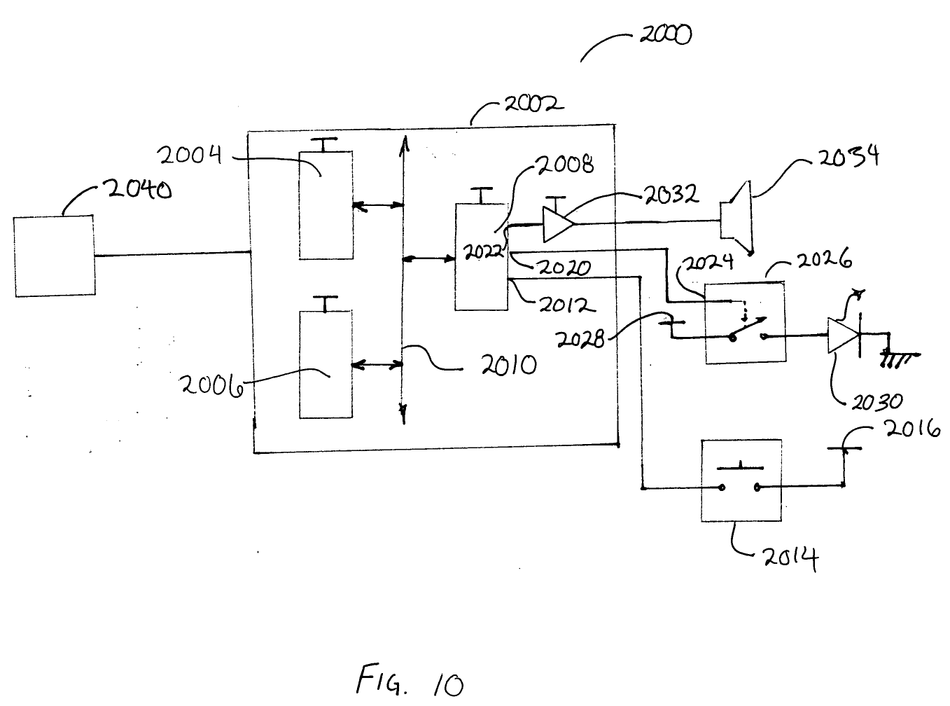

With the foregoing in mind, we now consider FIG. 10 which shows, in block diagram form, a linguistic audio system 2000 according to one embodiment of the invention.

As shown, the Linguistic audio system includes a voice generation portion 2002. In the illustrated embodiment, the voice generation portion includes a central processor unit 2004, a memory device 2006, and an input/output (I/O) device 2008. A communication bus 2010 is mutually coupled to respective communication ports of the central processor unit 2004, the memory device 2006 and the I/O device 2008. In one embodiment, the communication bus 2010 includes a data bus. In one embodiment, the communication bus 2010 includes a control bus. In one embodiment, the communication bus 2010 includes an instruction bus.

In the illustrated embodiment, the I/O device 2008 includes an input port 2012 coupled to, for example, an input switch 2014. As illustrated, the input switch is a normally open single pole pushbutton switch that when activated is adapted to couple a source of electrical potential 2016 to the input port 2012.

As illustrated, the I/O device 2008 also includes a first output port 2020 and a second output port 2022. The first output port 2020 is shown electrically coupled to a control input 2024 of a controlled switching device 2026 such as, for example, a transistor or an electromechanical relay. The controlled switching device is also coupled to, for example, a source of electrical current 2028, and to a load 2030 such as, for example, an illumination source.

As illustrated, the load 2030 is a light emitting diode that is adapted to be illuminated or extinguished in response to a state of a signal received at the control input 2024.

In the illustrated embodiment, output 2022 is coupled to an input of a digital to analog converter and amplifier (DACA) 2032. An output of the DACA 2032 is coupled to an input of a transducer 2034, such as a loudspeaker, a headphone set, or an ear bud.

Also shown is a power supply 2040. The power supply is adapted to be coupled to and supply electrical power to components of the system such as, for example, the central processor unit 2004, the memory device 2006, the I/O device 2008 and/or the DACA 2032.

In an exemplary operation, the system starts in a state in which the illumination source 2030 is not illuminated. An operator, by depressing the pushbutton 2014 applies an electrical signal to input 2012. A corresponding signal is received by the central processor unit 2004 from the I/O device across the communication bus 2010. The central processor unit 2004, executing one or more program steps that are stored, for example in memory device 2006, produces a responsive signal that is received over communication bus 2010 at I/O device 2008. I/O device 2008 produces a signal at output 2020 that is received at input 2024 of controlled switching device 2026.

In response, controlled switching device 2026 acts to couple the light emitting diode load 2030 to source of electrical current 2028, thereby powering the load 2030 and illuminating the light emitting diode.

In one embodiment, shortly after the light is illuminated (but effectively concurrently for practical purposes), the central processor unit 2004 retrieves pre-programmed data from the memory device 2006 for producing a voice output. The pre-programmed data may be recorded voice data, or voice synthesis data as discussed above in relation to FIG. 1.

Depending on whether the embodiment operates (or is currently operating) in a voice reproduction or voice synthesis mode, the central processing unit 2004 outputs the pre-programmed data to the I/O device 2008 or synthesizes a signal that is output to the I/O device 2008 by way of the communication bus 2010. The I/O device 2008, in turn, outputs the data received from the central processor unit 2004 to the DACA 2032, which outputs an analog signal to the audio transducer 2032. The audio transducer 2043 produces an analog voice signal coupled to ambient air. In the present example, the analog voice signal in the air might be perceived by a user as “lamp on.”

One of skill in the art will appreciate that the foregoing examples are merely illustrative, and are not intended to be comprehensive. For example, the linguistic audio system could include additional components such as, for example, a filter and an audio integrated circuit device, such as a voice chip, for example. Nevertheless, from the foregoing description and the attached claims, one of skill in the art will appreciate the broad scope of the present invention and the requisite detail for the implementation thereof.

1. A control system for dentistry applications comprises:
a built-in voice alert system comprising an electronic voice generating circuit having recorded voice messages applicable to a dental procedure; and
an electronic circuit programmed to play back any of the recorded voice messages when an appropriate stage is reached in the dental procedure.

2. The control system of claim 1 wherein said dental procedure comprises a dental whitening process, a dental curing process, or a root canal procedure.

3. The control system of claim 1 wherein said recorded messages comprises words for indicating the stage of completion of a whitening process, a curing process, a depth of reach in a root canal procedure, time intervals, or combinations thereof.

4. The control system of claim 1 wherein said system comprises an electronic timer device controlled by a microprocessor said timer device having an internal clock adapted for receiving a signal from a dental instrument to indicate the start of a dental process.
